(19) United States
(12) Patent Application Publication
(10) Pub. No.: US 2006/0034768 A1
(43) Pub. Date: Feb. 16, 2006

(54) Method and device for molecular imaging with the aid of molecular probe

(76) Inventor: Reiner Franz Schulz, Dormitz (DE)

Correspondence Address: HARNESS, DICKEY & PIERCE, P.L.C., P.O.BOX 8910, RESTON, VA, 20195, US

(21) Appl. No.: 11/200,075

(22) Filed: Aug. 10, 2005

(30) Foreign Application Priority Data
Aug. 11, 2004 (DE) 10 2004 039 048.7

(51) Int. Cl. A61K 49/04; G01T 1/00

(52) U.S. Cl. 424/9.42; 378/1

(57) ABSTRACT

A method is disclosed for molecular imaging with the aid of a molecular probe, the latter containing a contrast enhancing component that is detectable via X-ray fluorescence radiation emitted thereby. A device contains an X-ray source for radiating with X-radiation an object provided with the molecular probe, as well as an X-ray receiver for receiving an X-ray fluorescence radiation emitted by the contrast enhancing component.

The present application hereby claims priority under 35 U.S.C. §119 on German patent application number DE 10 2004 039 048.7 filed Aug. 11, 2004, the entire contents of which is hereby incorporated herein by reference.

### FIELD

The invention generally relates to a method and/or a device for molecular imaging with the aid of a molecular probe. The invention also generally relates to a molecular probe suitable for carrying out the method.

### BACKGROUND

Molecular imaging is a diagnostic imaging method with the aid of which it is possible to generate an image on the molecular or cellular plane. For this purpose, so-called molecular probes that are introduced into the object to be examined and are tuned to a specific pathological target are used. The molecular probes, tuned in such a way, attach themselves to this pathological target and are enriched in zones with corresponding pathological properties.

Such a molecular probe includes a carrier that reacts selectively to the respective pathological target and is in the form of a molecule or a nanoparticle that couples to receptors on cell surfaces that are characteristic of the pathological biochemical process to be detected. This carrier is provided with a diagnostic signal transmitter or to a contrast enhancing component enabling a detection of the molecular probe that is required for imaging.

The methods known in the prior art for detecting molecular probes are explained in more detail in “MEDICAMUNDI” 47/1, April 2003, pages 2 to 9, for example. One group of methods is based on nuclear medicine methods in the case of which a radio nuclide is used as contrast enhancing component. Although these methods have a high detection sensitivity, they exhibit the disadvantage that it is very complicated to produce and handle the required radio nuclides.

An alternative method of detection resides in detecting a suitable contrast enhancing component with the aid of magnetic resonance tomography. The units required for this purpose are, however, very expensive.

Another known method is based on detecting a fluorescence radiation in the near infrared. This method is certainly relatively cost effective and sensitive, but enables only the detection of molecular probes that are located in the vicinity of the surface of an object to be examined. However, even with this method the spatial resolution is not satisfactory, owing to the scattering of the infrared light inside the object.

### SUMMARY

At least one embodiment of the invention includes an object of specifying a method for molecular imaging with the aid of a molecular probe in the case of which high detection sensitivity is possible together with a low technical outlay. At least one embodiment of the invention also includes an object of specifying a device for carrying out the method and/or a molecular probe suitable for carrying out the method.

In the case of the inventive method for molecular imaging with the aid of a molecular probe, the latter contains a contrast enhancing component that is detected by way of X-ray fluorescence radiation. Such a method can be used to attain a high spatial resolution and detection sensitivity with a relatively low technical outlay.

A good detection sensitivity is attained when the contrast enhancing component includes an element whose atomic number is greater than 30. The probability of the occurrence of a fluorescence radiation is then greater than the probability of a competing Auger process.

In an advantageous refinement of at least one embodiment of the invention, use is made in the contrast enhancing component of an element whose atomic number is greater than 30, preferably greater than 55. In particular, the element used is from the group of lanthanides, in particular gadolinium Gd. As a result of this measure, the energy of the fluorescence quanta suffices for detecting the molecular probe even at relatively large depths inside a weakly absorbing object, for example in soft tissue parts or the mammary gland or, in particular, in the case of an atomic number that is greater than 55, in a more strongly absorbing matrix, for example in extremities with bones or in the body trunk.

A particularly high detection sensitivity is attained when the molecular probe contains at least 10⁴ atoms of the element.

In a particularly advantageous refinement of at least one embodiment of the method, use is made for the purpose of exciting the X-ray fluorescence radiation of an X-radiation whose spectral maximum is above the K edge of the element. This measure enhances or even ensures that the predominant fraction of the spectrum of the X-radiation can contribute to the excitation of an X-ray fluorescence radiation such that the radiation exposure of a patient or of the operating staff can be kept as low as possible. Particularly advantageous is a spectrum on the exciting X-radiation that emits virtually no or only a negligible number of X-ray quanta below the K edge of the relevant element such that the entire spectrum can be used diagnostically.

A device in accordance with at least one embodiment of the invention includes an X-ray source for irradiating with X-radiation an object provided with the molecular probe, and an X-ray receiver for receiving an X-ray fluorescence radiation emitted by the contrast enhancing component.

When the X-ray source generates X-radiation whose spectral maximum is above the K edge of an element serving as contrast enhancing component and, in particular, when a device for generating a narrowband X-ray spectrum is arranged downstream of the X-ray source, the efficiency of the excitation is particularly high since virtually the entire spectrum of the X-radiation directed on the object can generate fluorescence. Such a device can be, in particular, a crystal monochromator or a filter arrangement.

Alternatively or in addition thereto, the X-ray source has an anode that emits the X-ray tube with a narrowband X-radiation aimed at from the beginning.

The X-ray source preferably generates a collimated X-ray beam. This measure enables a high degree of spatial resolution even when use is made of a large-area detector, since the fluorescence radiation can come only from a spatial zone limited by the collimated X-ray beam. Since the detector can be of large area, it is able to detect this fluorescence radiation from a large solid angle such that the detection sensitivity is correspondingly raised.

A flat energy-discriminating detector is provided, in particular, as X-ray receiver. Owing to the selective detection of the fluorescence quanta, the background component is reduced and the detection sensitivity is correspondingly raised, in this way.

In an alternative refinement of at least one embodiment of the invention, an X-ray source is provided that generates an uncollimated X-ray beam. This measure shortens the measurement period, since the object is picked up over a large area by the excitation radiation.

In order to attain a high level of spatial resolution even with this refinement, a flat, spatially resolving and energy-discriminating detector is provided as X-ray receiver with an upstream collimator device.

A molecular probe particularly suitable for use in a method according to at least one embodiment of the invention contains at least 10⁴ atoms of an element that can be detected by X-ray fluorescence radiation.

### BRIEF DESCRIPTION OF THE DRAWINGS

Reference is made for the purpose of further explanation to the example embodiments of the drawings, in which:

FIG. 1 shows a device in accordance with an embodiment of the invention in a schematic illustrating principle,

FIGS. 2a, b respectively show different scanning possibilities for an object to be examined with the aid of a collimated X-ray beam,

FIG. 3 shows a diagram in which the mass absorption coefficient of gadolinium Gd is plotted together with an ideal and real excitation spectrum against the energy of X-ray quanta, and

FIGS. 4a, b respectively show a schematic of an arrangement in which an uncollimated X-ray beam is used to excite the X-ray fluorescence.

### DETAILED DESCRIPTION OF THE EXAMPLE EMBODIMENTS

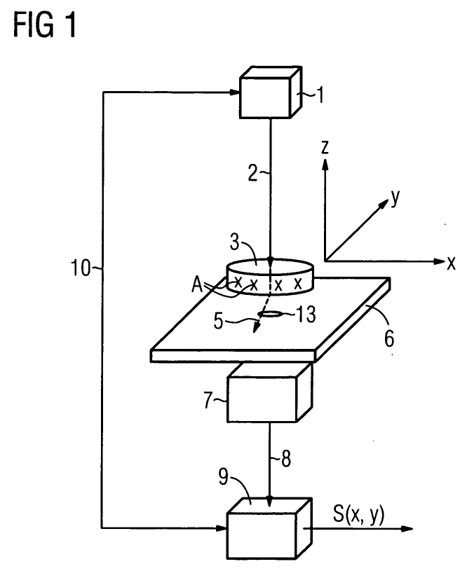

In accordance with FIG. 1, the device according to at least one embodiment of the invention includes an X-ray source 1 that emits an X-ray beam 2 that is used to irradiate an object 3 to be examined, for example a patient or a sample. Molecular probes A are introduced into the object 3 to be examined, and contain a contrast enhancing component that is excited to emit X-ray fluorescence radiation 5 by the X-ray beam 2. The X-ray fluorescence radiation 5 is detected by way of a large area X-ray receiver 6, for example a scintillator with a downstream photomultiplier. Arranged downstream of the X-ray receiver 6 is an electronic signal processing unit 7 in which measurement signals guided further by the X-ray receiver are processed and in particular discriminated in terms of their amplitudes. The object 3 to be examined is scanned by swiveling the X-ray source 1 or displacing it linearly.

Information 10 relating to the position of the X-ray source 1, that is to say knowledge of the position of the location at which the collimated X-ray beam 2 impinges on the object 3 to be examined is used in a central processor 9 together with the processed measurement signals 8 belonging to this location to generate an output signal S(x, y) that is a function of the location (x, y) and represents the distribution of the molecular probes A in the surface (x, y). Moreover, located between the object 3 to be examined and the X-ray receiver 6 is a beam stopper 13 for ensuring that the X-ray beam 2 emitted by the X-ray source 1 does not impinge directly on the X-ray receiver 6.

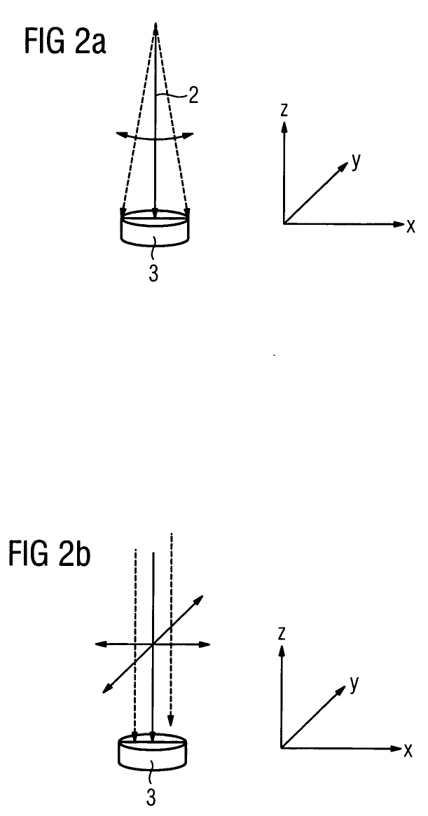

In accordance with FIG. 2a, the collimated X-ray beam 2 can scan the surface area—which is to be diagnostically acquired—of an object 3 to be examined, and does so—as illustrated by the double arrow in the figure—by swiveling movements about an axis in the zy-plane or zx-plane that is respectively parallel to the x-axis or y-axis.

In accordance with FIG. 2b, a translation in the x-direction or y-direction is also a possible alternative to this, as is illustrated in the figure by the two double arrows.

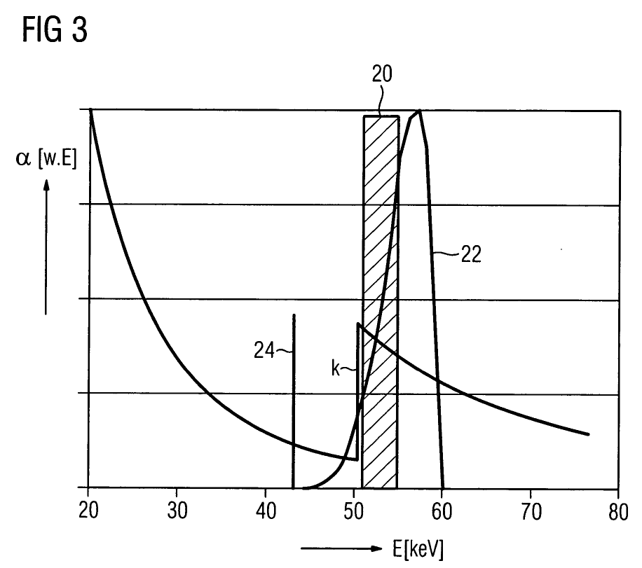

The mass absorption coefficient a of gadolinium Gd is plotted in arbitrary units (w.E.) in FIG. 3 against the energy E of the X-ray quanta. The K absorption edge K of gadolinium Gd is to be seen in the figure at about 50 keV. The use of an X-ray source that emits a relatively narrowband ideally rectangular excitation spectrum 20 above the K edge is expedient when use is made of a molecular probe A in which the contrast enhancing component includes gadolinium Gd as the element that can be detected by X-ray fluorescence spectroscopy. Such a narrowband excitation spectrum 20 can be generated at least approximately with the aid of a maximum output X-ray tube in combination with a crystal monochromator. Such an arrangement has the advantage that it is possible using one and the same X-ray source to set different mean excitation energies that are tuned to the element respectively included in the contrast enhancing component.

As an alternative to using a crystal monochromator, it is also possible to make use in the X-ray tube of anodes made from materials that exhibit a suitable characteristic radiation. Suitable anode material for gadolinium Gd as fluorescing element in the contrast enhancing component of the molecular probe A is, for example ytterbium Yb, which emits characteristic X-rays with energies of approximately 52 keV and 59 keV.

The desired narrowband excitation spectrum can also be generated with the aid of suitable filter materials. Depicted in the figure is an excitation spectrum 22 such as results given the use of an X-ray tube with a tungsten anode and a tungsten filter that is operated at a high voltage of 60 keV. Virtually all the excitation quanta are above the K edge of gadolinium Gd in this case, as well.

Combinations of the three above-named alternatives are also possible such that matched anode material, matched filter material and a monochromator come into use in combination. Given use of gadolinium Gd as the contrast enhancing element, it is possible to use ytterbium Yb as anode material or at least as an alloy constituent of the X-ray anode in combination with a crystal monochromator. A possible alternative to this is the use of hafnium Hf as anode material, which exhibits a characteristic X-radiation at 55.8 keV, in combination with a tungsten filter.

The figure also further depicts the significant K fluorescence line 24 of gadolinium Gd at approximately 43 keV.

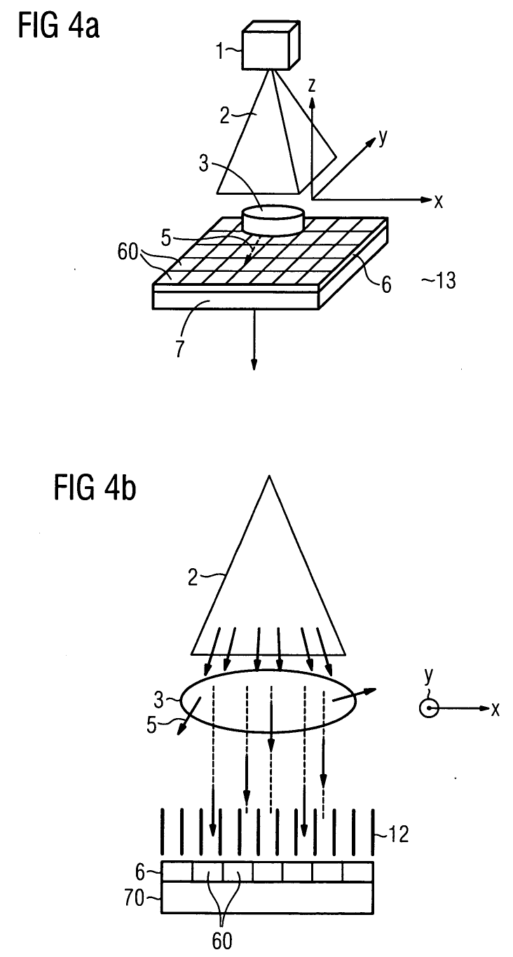

In accordance with FIGS. 4a, b, a conical X-ray beam 2 is emitted instead of a collimated X-ray beam. Such a conical divergent X-ray beam 2 can be used to completely detect a surface area to be examined of an object 3, without entailing the need of a relative movement between the object 3 and the X-ray source 1. Provided for the purpose of detecting the X-ray fluorescence radiation 5 is a flat spatially resolving energy-dispersive X-ray receiver 6 with a multiplicity of detector elements 60 that are arranged in the form of a two-dimensional receiver array and downstream of which a signal processing electronic system 70 is connected in each case. A directly converting absorber layer composed of a semiconductor, for example cadmium telluride CdTe or gallium arsenide GaAs is particularly suitable as detector element 60, each detector element 60 being coupled directly to a CMOS electronic system in which the measurement signals are simultaneously amplified and discriminated.

In order to improve the spatial resolution, it is advantageous in accordance with FIG. 4b to make use of a collimator device 12 which ensures that only fluorescence radiation 5 is detected which, as shown in the example of the figure, is propagated approximately parallel to the central axis of the X-ray 2 such that each detector element 60 of the two-dimensional receiver array can be uniquely assigned to a position in the x, y-plane (perpendicular to the plane of the drawing). In other words, each detector element 60 is assigned to a defined volumetric region of the object 3 to be examined that is approximately cuboid in the example.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for molecular imaging with the aid of a molecular probe, comprising:
detecting, via X-ray fluorescence radiation, an emission of the molecular probe containing a contrast enhancing component.

2. The method as claimed in claim 2, wherein the contrast enhancing component includes an element whose atomic number is greater than 30.

3. The method as claimed in claim 1, wherein the contrast enhancing component includes an element whose atomic number of the element is greater than 55.

4. The method as claimed in claim 3, wherein the element belongs to the group of lanthanides.

5. The method as claimed in claim 2, wherein the molecular probe contains at least 10⁴ atoms of the element.

6. The method as claimed in claim 2, wherein use is made of an X-radiation, for the purpose of exciting the X-ray fluorescence radiation, whose spectral maximum is above the K edge of the element.

7. A device for molecular imaging with the aid of a molecular probe that contains a contrast enhancing component detectable via X-ray fluorescence radiation, comprising:
an X-ray source for irradiating an object provided with the molecular probe with X-radiation; and
an X-ray receiver for receiving an X-ray fluorescence radiation emitted by the contrast enhancing component.

8. The device as claimed in claim 7, wherein the X-ray source generates X-radiation whose spectral maximum is above the K edge of an element serving as contrast enhancing component.

9. The device as claimed in claim 7, further comprising a device for generating a narrowband X-ray spectrum.

10. The device as claimed in claim 9, further comprising a crystal monochromator downstream of the X-ray source for the purpose of generating a narrowband X-ray spectrum.

11. The device as claimed in claim 9, further comprising a filter arrangement downstream of the X-ray source for the purpose of generating a narrowband X-ray spectrum.

12. The device as claimed in claim 7, wherein the X-ray source has an X-ray tube with an anode that emits narrowband X-radiation.

13. The device as claimed in claim 7, wherein the X-ray source generates a collimated X-ray beam.

14. The device as claimed in claim 13, wherein a flat energy-discriminating detector is provided as X-ray receiver.

15. The device as claimed in claim 7, wherein the X-ray source generates an uncollimated X-ray beam.

16. The device as claimed in claim 15, wherein a flat, spatially resolving and energy-discriminating detector is provided as X-ray receiver with an upstream collimator device.

17. A molecular probe for molecular imaging, which contains a contrast enhancing component having at least 10⁴ atoms of an element that is detectable via X-ray fluorescence radiation emitted thereby.

18. The molecular probe as claimed in claim 17, wherein the contrast enhancing component includes an element whose atomic number is greater than 30.

19. The molecular probe as claimed in claim 18, wherein the atomic number of the element is greater than 55.

20. The molecular probe as claimed in claim 19, wherein the element belongs to the group of lanthanides.
